(19) United States
(12) Patent Application Publication
(10) Pub. No.: US 2006/0147437 A1
(43) Pub. Date: Jul. 6, 2006

(54) Use of pigmented retinal epithelial cells for creation of an immune privilege site

(76) Inventors: Richard C. Allen, Flemington, NJ (US); Michael L. Cornfeldt, Morristown, NJ (US); William R. Kidwell, Ijamsville, MD (US)

Correspondence Address: MORRISON & FOERSTER LLP, 755 PAGE MILL RD, PALO ALTO, CA, 94304-1018, US

(21) Appl. No.: 11/363,911

(22) Filed: Feb. 27, 2006

Related U.S. Application Data
(63) Continuation of application No. 09/002,413, filed on Jan. 2, 1998, now abandoned.

(51) Int. Cl. A61K 48/00

(52) U.S. Cl. 424/93.21

(57) ABSTRACT

The present invention relates to a novel in vivo method for creation of a localized immunosuppressive environment in tissue. The method involves the transplanting of pigmented retinal epithelial cells into a mammal thereby producing a localized immunosuppressive environment. The transplanted pigmented retinal epithelial cells may also be used to produce therapeutic proteins or other biologically active molecules that may be useful in treatment of diseases.

### 1. INTRODUCTION

The present invention relates to a novel in vivo method for creation of a localized immunosuppressive environment in tissue. The method involves the transplanting of pigmented retinal epithelial cells into a mammal thereby producing a localized immunosuppressive environment. The transplanted pigmented retinal epithelial cells may also be used to produce therapeutic proteins or other biologically active molecules that may be useful in treatment of diseases.

### 2. BACKGROUND OF THE INVENTION

Certain chronic diseases result in the destruction of functional cells in affected organs. Mammals with such diseases are frequently unable to produce proteins or hormones necessary to maintain normal physiological function. In such instances, transplantation of healthy organs or cells into the affected mammal may alleviate the symptoms of the disease. The transplantation of cells and tissues is being utilized therapeutically in a wide range of disorders including but not limited to cystic fibrosis (lungs), kidney failure, degenerative heart diseases, diabetes, neurodegenerative disorders, liver failure and pancreatic failure.

Unfortunately, such transplants are often rejected by the body due to an immune response initiated in response to the foreign tissue or cells. Presently, the only recourse to prevent the rejection of the transplanted tissue is to administer immunosuppressive agents, but the individual is placed at medical risk making the immunosuppressant therapy itself more of a liability than a benefit in some cases. Therefore, the benefits of transplantation have been limited by the serious side effects of systemic immunosuppression, which is necessary if successful transplantation is to be achieved in humans.

It has recently been discovered that immune-privileged sites exist in the body where grafted tissue can survive for prolonged periods of time (Streilan, J. W., 1995, Science 270:1158-1159). Such sites include, for example, the eye, testes, and brain. The features of the privileged sites include intratissue structural barriers such as the presence of a blood-tissue barrier, absence of efferent lymphatics and direct drainage of tissue fluid into the blood. Additional features of immune privileged sites include the establishment of an immunosuppressive environment through secretion of immunosuppressive cytokines such as TGF β or Fas L. The Fas L protein is believed to be particularly important for the prolonged survival of grafted tissue and is believed to act through activation of apoptosis in Fas+, antigen activated T cells of the recipient (Griffith, T. S. et al., 1995, Science 270:1189-1192).

The eye, an organ segregated into two anatomically distinct regions, is a particularly interesting example of an immune privileged site. The immune privilege in the anterior chamber is believed due to Fas L, while that in the posterior chamber is believed due to the physical barrier created by the RPE cells of the retina, segregating the posterior chamber from the immune cells of the blood. Based on this, it would be surprising indeed if isolated RPE cells, no longer in a tight confluent layer, could produce an immune privileged site.

The present invention is based on the discovery that retinal pigmented epithelial cells secrete Fas L and are capable of functioning outside of the structural confines of the retina to produce an immune privileged site. The expression of Fas L protein by retinal pigmented epithelial cells is surprising given the fact that they also express the receptor for Fas L (Esser, et al., 1995, Bioch. Biophys. Res. Com. 213:1206-1034). Nevertheless, the cells seem resistant to the signals for apoptosis.

The present invention is based on discovery that human retinal epithelial cells secrete the Fas L protein. Expression of Fas L in the immune-privileged site of the eye, is believed to directly kill activated lymphocytes that might invade the eye in response to inflammation and thereby destroy vision by reacting with important structures such as the retina. The expression of the Fas L protein by retinal epithelial cells is surprising given the fact that the human retinal epithelial cells also express the receptor for Fas L (Esser et al., 1995, Bioch. Biophys. Res. Com. 213:1026-1034). Nevertheless, the cells seem resistant to the signals for apoptosis.

Recently, studies have suggested that Sertoli cells, when simultaneously transplanted with pancreatic islet cell into the diabetic rat, act as an effective local immunosuppressant on the host tissue (Selawry and Cameron, 1993, Cell Transplantation 2:123-129). This cell transplantation protocol is accomplished without prolonged systemic immunosuppression, otherwise necessary when islets are transplanted without Sertoli cells. As a result, the graft is not rejected and the islets remain viable allowing the transplanted pancreatic islet cells to function normally and produce insulin for an indefinite period of time. Survival of the graft seems to correlate with constitutive expression of Fas L by the Sertoli cells.

The development of methods designed to enhance productive cell transplantation techniques would be useful for the treatment of diseases, such as Parkinson's disease, and diabetes. Likewise, it is desirable to avoid systemic immunosuppression with the ability to locally immunosuppress (i.e., at the graft site) by administration of an immunosuppressant that is biologically tolerated by the host. Therefore, the identification of cells capable of delivering local immunosuppression and promoting efficient graft acceptance and functional restoration of the tissue-related dysfunction is desirable.

### 3. SUMMARY OF THE INVENTION

The present invention relates to a novel method for creation of an immunologically privileged site in a mammal. The method of the invention comprises the transplantation of retinal pigment epithelial (RPE) cells, thereby producing a localized immunosuppressive environment at the site of transplantation. The present invention relates to the discovery that RPE cells secrete large quantities of the immunosuppressive cytokine referred to as Fas-Ligand (Fas L). The Fas L protein is believed to exert its immune suppressive effect by stimulating apoptosis in Fas+ antigen activated T cells of the recipient. In addition to immunosuppressive cytokines, the RPE cells produce additional biological factors such as growth factors, cytokines, and hormones that may be useful in treating a wide range of different diseases.

The invention further relates to the co-administering of RPE cells together with cells that supply a functionally active therapeutic molecule as a method of treating diseases resulting from a deficiency of a biological factor in a mammal. In instances where the RPE cells are co-administered with cells and/or matrices supplying therapeutic molecules, the RPE cells may be co-administered either as a single composition, or alternatively, as separate compositions. When the RPE cells are administered as a separate composition, the RPE cells may be administered prior to co-administration of cells that supply a therapeutic, protein or biologically active molecule, in a sufficient amount for creation of an immune privilege site. The co-administering of RPE cells has the advantage in that the RPE cells create an immunologically privileged site thereby increasing the survival time of the co-administered cells. Co-administered cells producing functionally active proteins or biologically active molecules, include but are not limited to, insulin producing β-cells, dopamine producing neural or non-neural cells or hormone producing endocrine cells.

In yet another embodiment of the invention, RPE cells may be genetically engineered to produce a therapeutic protein or biologically active molecule that may be useful in treating disease. For example, the RPE cells may be genetically engineered to produce a wide range of proteins including but not limited to, growth factors, cytokines, or biologically active molecules such as hormones. The ability of RPE cells to suppress the normal graft rejection response ordinarily stimulated in the recipient host increases the growth and viability of the transplanted RPE cells. The invention further relates to the in vitro attachment of RPE cells to the same or different matrix for the purpose of increasing the long term viability of the transplanted cells. In addition, co-administered cells producing therapeutic proteins or biologically active molecules, may be attached to the same or different matrix prior to transplantation. Materials of which the support matrix can be composed include those material to which cells adhere following in vitro incubation, on which cells can grow, and which can be implanted into the mammalian body without producing a toxic reaction, or an inflammatory reaction which would destroy the implanted cells.

The invention provides for pharmaceutical compositions comprising RPE cells and a pharmaceutically acceptable carrier. The invention further encompasses pharmaceutical compositions comprising RPE cells and cells producing a functionally active therapeutic protein, or biologically active molecule, contained in a pharmaceutically acceptable carrier. The compositions of the invention may be utilized for treatment of diseases where the creation of an immunologically privileged site and the administration of a functionally active therapeutic protein, or other biologically active molecule, is desired. Such diseases include neurological, cardiac, endocrine, hepatic, pulmonary, metabolic or immunological related diseases. For example, neurological disorders such as Parkinson's disease, Huntington's disease, Alzheimer's disease, ALS, stroke and traumatic head and spinal injury may be treated. Non-neurological diseases include, but are not limited to, diabetes, blood clotting disorders, and cystic fibrosis.

### 4. BRIEF DESCRIPTION OF THE FIGURES

FIG. 1. FACS analysis of Fas L induced apoptosis. The presence of apoptotic cells is demonstrated by increased fluorescence intensity. The percent of apoptotic cells increases in proportion to the level of Fas L present in the media.

FIG. 2. FACS analysis of Fas L induced apoptosis. Increased apoptosis in the presence of Fas L is indicated in the accompanying table inserts presented below each FACS analysis.

### 5. DETAILED DESCRIPTION OF THE INVENTION

The present invention provides a method of producing a sustained localized immunosuppressive effect in tissue. This is achieved by the general step of transplanting RPE cells into host recipient tissue. By sustained localized immunosuppressive effect, it is meant that the transplanted RPE cells will suppress the immunological response ordinarily mounted by the host tissue to foreign entities such as transplanted cells and that the immunosuppression will occur at the graft site (local) rather than by generalized immunosuppression of the entire body (systemic) which occurs with the ordinary methods of immunosuppression by agents such as cyclosporine.

In a preferred embodiment, the transplanted RPE cells (which are intended to replace dysfunctional cells or in some way alleviate tissue dysfunction) can avoid being rejected and thereby survive and functionally integrate into the host tissue. Furthermore, the method of the present invention can also be utilized wherein RPE cells are co-administered with additional cells or tissues, such as neural cells, endocrine cells, muscle cells, and other cells that produce a functionally active therapeutic molecules. In addition, the RPE cells may be attached in vitro prior to transplantation to a natural or synthetic matrix that increases the long term viability of the transplanted cells. The method of the present invention may be used for enhancing the outcome of tissue transplants, by providing localized immunosuppression. That is, RPE cells may be used to facilitate transplant survival and graft function of the cells being transplanted.

The present invention is based on the discovery that RPE cells secrete the immunosuppressive cytokine Fas L. The Fas L protein has been shown to prolong the viability of grafted tissue through activation of apoptosis in Fas+ antigen activated lymphocytes of the recipient.

With local immunosuppression by a RPE cell-derived immunosuppressant agent, such as Fas L, there would be no cellular immunological attack waged against the transplanted cells, including the RPE cells themselves. Additionally, since the immunosuppression is local and by a biologically tolerable agent, the side effects associated with both systemic immunosuppression and cytotoxicity of agents such as cyclosporine would be avoided. Hence, the method of RPE cell transplantation provides a significant improvement over the use of systemic immunosuppression with cyclosporine as the necessary adjunctive therapy to transplantation.

The localized immunosuppression by a RPE cell-derived immunosuppressant agent, such as Fas L, can facilitate the survival of both xenografts and allografts. With allografts, co-transplantation with RPE cells should provide localized immunosuppression as to eliminate the need for systemic immunosuppression. With xenografts, co-transplantation with RPE cells may provide sufficient local immunosuppression so as to eliminate the need for systemic immunosuppression or the RPE cells may be used in combination with a systemic immunosuppressant to prevent rejection thereby reducing the dosage of systemic immunosuppressant required. When co-transplanted, the RPE cells may not only provide immunosuppression, but may provide regulatory, nutritional, and other factors which support the survival and/or growth of co-transplanted tissue. Therefore, the RPE cells will not only provide inhibition of the immune response, but will allow enhanced growth and viability of allografts and xenografts by concomitant trophic support.

### 5.1. Sources of RPE Cells

The source of RPE cells is by primary cell isolation from the mammalian retina. Protocols for harvesting RPE cells is well-defined (Liu and Turner, 1988, Exp. Eye Res 47:911-917; Lopez et al., 1989, Invest Ophthalmol Vis Sci. 30:586-588) and considered a routine methodology (see below, Section 6.6.1.). In most of the published reports of RPE cell co-transplantation, cells are derived from the rat (Liu and Turner, 1988, Exp. Eye Res 47:911-917; Lopez et al., 1989, Invest Ophthalmol Vis Sci. 30:586-588), although, it is contemplated that the method of the present invention can be used with RPE cells from any suitable mammalian source. A preferred source of RPE cells for use with mammals, such as humans, are human RPE cells. However, if available and suitable, porcine RPE cells may be utilized. In addition, to isolated primary RPE cells, cultured human and animal RPE cell lines may be used in the practice of the invention. The methods of the invention further encompass the transplantation of RPE cells genetically engineered to express functionally active therapeutic proteins, enzymes that produce biologically active factors or biologically active molecules.

The present methods and compositions may employ RPE cells genetically engineered to produce a wide range of functionally active therapeutic proteins, enzymes that produce biologically active factors or biologically active molecules including growth factors, cytokines, hormones and peptide fragments of hormones, inhibitors of cytokines, peptide growth and differentiation factors, interleukins, chemokines, interferons, colony stimulating factors and angiogenic factors. Examples of such proteins include, but are not limited to, the superfamily of TGF-β molecules, including the five TGF-β isoforms and bone morphogenetic proteins (BMP), latent TGF-β binding proteins, LTBP; keratinocyte growth factor (KGF); hepatocyte growth factor (HGF); platelet derived growth factor (PDGF); insulin-like growth factor (IGF); the basic fibroblast growth factors (FGF-1, FGF-2 etc.), vascular endothelial growth factor (VEGF); Factor VIII and Factor IX; erythropoietin (EPO); tissue plasminogen activator (TPA); activins and inhibins. Hormones which may be used in the practice of the invention include growth hormone (GH) and parathyroid hormone (PTH).

One may obtain the DNA segment encoding the protein of interest using a variety of molecular biological techniques, generally known to those skilled in the art. For example, cDNA or genomic libraries may be screened using primers or probes with sequences based on the known nucleotide sequences. Polymerase chain reaction (PCR) may also be used to generate the DNA fragment encoding the protein of interest. Alternatively, the DNA fragment may be obtained from a commercial source.

The DNA encoding the translational or transcriptional products of interest may be recombinantly engineered into variety of vector systems that provide for replication of the DNA in large scale for the preparation of genetically engineered RPE cells. These vectors can be designed to contain the necessary elements for directing the transcription and/or translation of the DNA sequence in RPE cells.

Vectors that may be used include, but are not limited to those derived from recombinant bacteriophage DNA, plasmid DNA or cosmid DNA. For example, plasmid vectors such as pBR322, pUC 19/18, pUC 118, 119 and the M13 mp series of vectors may be used. Bacteriophage vectors may include λgt10, λgt11, λgt18-23, λZAP/R and the EMBL series of bacteriophage vectors. Cosmid vectors that may be utilized include, but are not limited to, pJB8, pCV 103, pCV 107, pCV 108, pTM, pMCS, pNNL, pHSG274, COS202, COS203, pWE15, pWE16 and the charomid 9 series of vectors. Alternatively, recombinant virus vectors including, but not limited to those derived from viruses such as herpes virus, retroviruses, vaccinia viruses, adenoviruses, adeno-associated viruses or bovine papilloma virus may be engineered.

Methods which are well known to those skilled in the art can be used to construct expression vectors containing the protein coding sequence operatively associated with appropriate transcriptional/translational control signals. These methods include in vitro recombinant DNA techniques, and synthetic techniques. See, for example, the techniques described in Sambrook, et al., 1992, Molecular Cloning, A Laboratory Manual, Cold Spring Harbor Laboratory, N.Y. and Ausubel et al., 1989, Current Protocols in Molecular Biology, Greene Publishing Associates & Wiley Interscience, N.Y.

The genes encoding the proteins of interest may be operatively associated with a variety of different promoter/enhancer elements. The expression elements of these vectors may vary in their strength and specificities. Depending on the host/vector system utilized, any one of a number of suitable transcription and translation elements may be used. The promoter may be in the form of the promoter which is naturally associated with the gene of interest. Alternatively, the DNA may be positioned under the control of a recombinant or heterologous promoter, i.e., a promoter that is not normally associated with that gene. For example, RPE specific promoter/enhancer elements may be used to regulate the expression of the transferred DNA in RPE cells.

In some instances, the promoter elements may be constitutive or inducible promoters and can be used under the appropriate conditions to direct high level or regulated expression of the gene of interest. Expression of genes under the control of constitutive promoters does not require the presence of a specific substrate to induce gene expression and will occur under all conditions of cell growth. In contrast, expression of genes controlled by inducible promoters is responsive to the presence or absence of an inducing agent.

Specific initiation signals are also required for sufficient translation of inserted protein coding sequences. These signals include the ATG initiation codon and adjacent sequences. In cases where the entire coding sequence, including the initiation codon and adjacent sequences are inserted into the appropriate expression vectors, no additional translational control signals may be needed. However, in cases where only a portion of the coding sequence is inserted, exogenous translational control signals, including the ATG initiation codon must be provided. Furthermore, the initiation codon must be in phase with the reading frame of the protein coding sequences to ensure translation of the entire insert. These exogenous translational control signals and initiation codons can be of a variety of origins, both natural and synthetic. The efficiency and control of expression may be enhanced by the inclusion of transcription attenuation sequences, enhancer elements, etc.

It is also within the scope of the invention that multiple genes, combined on a single genetic construct under control of one or more promoters, or prepared as separate constructs of the same or different types may be used. Thus, an almost endless combination of different genes and genetic constructs may be employed. Certain gene combinations may be designed to, or their use may otherwise result in, achieving synergistic effects on cell stimulation any and all such combinations are intended to fall within the scope of the present invention. Indeed, many synergistic effects have been described in the scientific literature, so that one of ordinary sill in the art would readily be able to identify likely synergistic gene combinations, or even gene-protein combinations.

For long-term, high-yield production of recombinant proteins, stable expression is preferred. Rather than using expression vectors which contain viral origins of replication, host RPE cells can be transformed with DNA controlled by appropriate expression control elements (e.g., promoter, enhancer sequences, transcription terminators, polyadenylation sites, etc.), and a selectable marker. Following the introduction of the foreign DNA, engineered RPE cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express a therapeutic gene product of interest.

To increase the long term viability of the transplanted cells, i.e., transplanted RPE cells or co-administered cells, the cells to be transplanted can be attached in vitro to a support matrix prior to transplantation. Materials of which the support matrix can be comprised include those materials to which cells adhere following in vitro incubation, and on which cells can grow, and which can be implanted into the mammalian body without producing a toxic reaction, or an inflammatory reaction which would destroy the implanted cells or otherwise interfere with their biological or therapeutic activity. Such materials may be synthetic or natural chemical substances or substances having a biological origin. The matrix materials include, but are not limited to, glass and other silicon oxides, polystyrene, polypropylene, polyethylene, polyvinylidene fluoride, polyurethane, polyalginate, polysulphone, polyvinyl alcohol, acrylonitrile polymers, polyacrylamide, polycarbonate, polypentent, nylon, amylases, natural and modified gelatin and natural and modified collagen, natural and modified polysaccharides, including dextrans and celluloses (e.g. nitrocellulose), agar, and magnetite. Either resorbable or non-resorbable materials may be used. Also intended are extracellular matrix materials, which are well-known in the art. Extracellular matrix materials may be obtained commercially or prepared by growing cells which secrete such a matrix, removing the secreting cells, and allowing the cells which are to be transplanted to interact with and adhere to the matrix. The matrix material on which the cells to be implanted grow, or with which the cells are mixed, may be an indigenous product of the RPE cells themselves. Thus, for example, the matrix material may be extracellular matrix or basement membrane material which is produced and secreted by the RPE cells to be implanted.

To improve cell adhesion, survival and function, the solid matrix may optionally be coated on its external surface with factors known in the art to promote cell adhesion, growth or survival. Such factors include cell adhesion molecules, extracellular matrix, such as, for example, fibronectin, laminin, collagen, elastin, glycosaminoglycans, or proteoglycans or growth factors, such as, for example, nerve growth factor (NGF). Alternatively, if the solid matrix to which the implanted cells are attached is constructed of porous material, the growth- or survival-promoting factor or factors may be incorporated into the matrix material, from which they would be slowly released after implantation in vivo.

When attached to the support according to the present invention, the cells used for transplantation are generally on the “outer surface” of the support. The support may be solid or porous. However, even in a porous support, the cells are in direct contact with the external milieu without an intervening membrane or other barrier. Thus, according to the present invention, the cells are considered to be on the “outer surface” of the support even though the surface to which they adhere may be in the form of internal folds or convolutions of the porous support material which are not at the exterior of the particle or bead itself.

The configuration of the support is preferably spherical, as in a bead, but may be cylindrical, elliptical, a flat sheet or strip, a needle or pin shape, and the like. A preferred form of support matrix is a glass bead. Another preferred bead is a polystyrene bead. Bead sizes may range from about 10 microns to 1 mm in diameter, preferably from about 90 to about 150 μm. For a description of various microcarrier beads, see, for example, Fisher Biotech Source 87-88, Fisher Scientific Co., 1987, pp. 72-75; Sigma Cell Culture Catalog, Sigma Chemical Co., St. Louis, 1991, pp. 162-163; Ventrex Product Catalog, Ventrex Laboratories, 1989; these references are hereby incorporated by reference. The upper limit of the bead's size may be dictated by the bead's stimulation of undesired host reactions, which may interfere with the function of the transplanted cells or cause damage to the surrounding tissue. The upper limit of the bead's size may also be dictated by the method of administration. Such limitations are readily determinable by one of skill in the art.

### 5.2. Pharmaceutical Formulations and Methods of Creating an Immunologically Privileged Site

The present invention encompasses methods and compositions for creating a localized immunosuppressive environment. Pharmaceutical compositions for use in accordance with the present invention may be formulated in conventional manner using one or more physiologically acceptable carriers or excipients. Thus, the RPE cells and any cells, tissue or matrices to be co-transplanted with the RPE cells, and physiologically acceptable salts and solvents may be formulated for administration by surgical transplantation or injection. As used herein, a pharmaceutically acceptable carrier includes any and all solvents, dispersion media, coatings, antibacterial and antifungal agents, isotonic agents and the like. The use of such media and agents is well-known in the art.

The present invention also encompasses compartmentalized kits adapted to receive a container adapted to contain RPE cells and a second container adapted to contain cells that produce a therapeutic molecule. The invention also relates to an article of manufacture comprising a packaging material and RPE cells contained within the packaging material.

The methods of the present invention, encompass the administration of RPE cells into a mammal so as to become located in proximity to the selected tissue. For example, the location can be any site within the mammalian body such as endothelial tissue, muscle tissue, neural tissue and organs, etc. The proximity of the RPE cells to the tissue is determined by the specific tissue being transplanted and the function sought to be restored in a given transplantation.

The administration of RPE cells is accomplished by conventional techniques. Preferred techniques for administration of RPE cells includes injection of RPE cells within the host or surgical transplantation of cells within the host. Prior to transplantation, the recipient mammal may be anesthetized using local or general anesthesia according to conventional techniques.

The number of RPE cells needed to achieve the purposes of the present invention will vary depending on the specific tissue being transplanted and the desired function of the RPE cells. For example, the RPE cells are administered in an amount effective to provide an immunologically privileged site. In general, such an effective amount is defined as that which prevents immune rejection of subsequently or coadministered cells or tissue. The dose range of RPE cells to be used in the practice of the invention may vary between 10³-10⁹ cells, although the preferable dose of administered RPE cells will be between 10⁵-10⁷ cells. Immune rejection can be determined for example histologically, or by functional assessment of the cotransplanted cells or tissue.

In an embodiment of the invention comprising the co-administration of cells producing a functionally active therapeutic protein or other biologically active molecule, together with RPE cells, the cells are administered in a therapeutically affective amount. In such an embodiment of the invention, the RPE cells may be co-administered as a single composition, or alternatively, as two separate compositions. Further, the RPE cells may be re-administered in an effective amount as necessary to sustain an immunologically privilege site. Alternatively, the co-administered cells that supply a therapeutic protein, or other biologically active molecule, may be re-administered in an effective amount to sustain a therapeutic effect.

In yet another embodiment of the invention, the transplanted cells may be attached in vitro to a matrix prior to transplantation. The number of cells to be transplanted can be determined by one of skill in the art by methods known in the art and will be dependent upon the amount of therapeutic protein or other biologically active molecule being produced by the cells and the known therapeutically effective amount of molecule necessary to treat the disease.

### 6. EXAMPLE

### Production of Immunologically and Biologically Active Fas L by RPE Cells

The section below describes experimental results demonstrating that retinal pigmented epithelial cells express biologically active Fas L. Enzyme linked immunoassays with anti-Fas L antibody indicated that substantial amounts of Fas L was released into the culture media by the retinal pigmented epithelial cells. In addition, the secreted Fas L was biologically active in inducing apoptosis in human fetal thymocytes.

### 6.1. Materials and Methods

### 6.1.1. Isolation and Culture of Retinal Pigmented Epithelial Cells

Primary isolates of RPE cells were made using human fetal human eyes at 18-20 weeks of gestation. Fetal eyes are collected within 15 minutes of harvesting the conceptus and their external surface is briefly washed with cold, sterile saline solution to remove as much external contamination as possible. The eye tissue is transferred into a dissecting dish containing solution A (RPMI 1640 culture media (Gibco, Cat. No. 22-400) to which a penicillin/streptomycin/fungizone Stock Solution (Gibco, Cat. No. 15240-039) is added to give a final concentration of 2% vol./vol.

Using sterile forceps and scissors, excess fat tissue is trimmed from the eye tissue. Using a sterile, disposable scalpel, the eye tissue is sectioned just behind the iris and the frontal tissue discarded. The back ⅔ of eye tissue is sectioned from top to bottom with the scalpel and the inner faces of the two halves oriented face up. Each half is then affixed to the silicone layer in the bottom of the Dissecting Dish using 3-4 one inch, sterile, disposable 23 gauge needles (Baxter, Cat. No. 23G1). This exposes the pigmented retinal epithelial cell layers, which are gently teased away from the choroid membrane to which the RPE cell sheet is attached. Usually, two large sheets of RPE cells are recovered from each eye.

Once the RPE cell layer is detached, it is examined microscopically to determine if there is significant contamination with choroid membrane. The RPE cell layer is transferred from the dish into 10 ml of sterile Solution A. Sterile filtered collagenase (Liberase™, Boehringer Mannheim) is added to a final concentration of 1 mg/ml. RPE tissue is transferred to a 37° C. water bath and incubated for 15 minutes. The tube is then centrifuged at 100×g for 5 min at room temperature in a Beckman bench top centrifuge (Beckman, Model No. GPR). The tube is transferred back to the laminar flow hood and the aqueous phase gently aspirated. Ten ml of Culture Medium (RPMI 1640 containing 10% fetal calf serum, 2 mM glutamine, and acidic FGF, 10 ng/ml) is added and the RPE tissue in the pellet resuspended. A small aliquot of the suspension is placed on a microscope slide and examined microscopically. The collagenase digestion step produces a limited fragmentation of the RPE cell sheath and removes the small residual choroid tissue and associated cell contaminants, but does not result in a dissociation of the RPE cell layer into single cells.

RPE cells derived as described above were suspended in 10 ml of Culture Medium to which Stock Solution of antibiotic/antimycotics added to a final concentration of 1%. All culture reagents (medium, serum, FGF, glutamine and the trypsin utilized for subculturing) have been qualified for GMP cell manufacturing by Washington Labs. These Qualified reagents are supplied by Washington Labs for the initial phase of cell expansion of primary isolates of RPE tissue. The RPE cell suspension is transferred to 25 ml Falcon culture flasks that are coated with a recombinant attachment protein, Pronectin F (Protein Technologies, Cat. No. 5002-00, Lot. No. RO117-c), to facilitate cell attachment.

Flasks are coated as follows: a 5 mg vial of sterile Pronectin F was dissolved in 5 ml of sterile diluent solution (lithium perchlorate in water) in a laminar flow hood. Aliquots are mixed with qualified Phosphate Buffered Saline (PBS) (Gibco, Cat. No. 14287) to produce a Pronectin F concentration of 10 μg/ml. Five ml of this solution is sterilely transferred into the Falcon culture flasks, which were allowed to stand in the laminar flow hood for two hours at room temperature. The solution is removed with a sterile pipet and the flask rinsed twice with sterile Pronectin F-free PBS. The flasks were allowed to dry in the laminar flow hood after removal of the second rinse solution. The caps are tightened on the flasks and the flasks stored under refrigeration for up to 4 months for RPE cell culture.

The Pronectin-F coating facilitates cell division by a factor of 4-5 fold, compared to that seen with uncoated flasks. Results seen with Pronectin F are approximately equivalent to those seen with mouse laminin (Gibco, Cat. No. L2020) and human laminin (Sigma, placental derived, Cat. No. L6274) coated culture flasks.

The initial culture of RPE cells is performed in Culture Medium to which Stock Solution antibiotic/antimycotic solution supplement is added to a final concentration of 1%. The cultures uniformly become contaminated by microbial agents that are acquired by the tissue during transit through the birth canal, if the antibiotic/antimycotic solution supplement is added to a final concentration of 1%. The cultures uniformly become contaminated by microbial agents that are acquired by the tissue during transit through the birth canal, if the antibiotic/antimycotic supplement is omitted from the Culture Medium. The antibiotic/antimycotic agents are maintained in the RPE cultures for approximately two weeks, with medium changes at least once weekly. Thereafter, the cultures are switched to antibiotic/antimycotic-free Culture Medium for an additional two weeks. Less than one culture in 10 presents evidence of contamination with bacteria, yeast or fungus after the shift to antibiotic/antimycotic-free medium, provided the antibiotic/antimycotic reagent is present from the time of tissue initiation.

The frequency of medium changes during the RPE cell culture is dictated by changes in glucose and lactate in the cultures. Following the initial plating of RPE cells, aliquots of medium are removed from the flasks once every two-three days and subjected to glucose and lactate analysis, using a YSI glucose-lactate analyzer (YSI, Model No. 2700). The analyzer is standardized at each assay using internal standards of glucose and lactate provided by YSI. If the analysis indicates that the cultures have consumed more than ½ to ⅔ of the glucose, the culture medium is changed. As a minimum, the culture medium is changed once weekly, to assure that effective concentrations of the antibiotic/antimycotic agents are maintained.

A comparison of glucose consumed to lactate produced is also determined. Uninfected culture medium exhibited a glucose:lactate ratio of 0.80:1 and greater in sparsely populated to near confluent cultures. Excessive lactate production by sparse cultures is viewed as an indication of contamination with bacteria and such cultures are discarded. Excessive consumption of glucose in the absence of approximately equivalent lactate accumulation is viewed as an indication of fungal or yeast contamination and such cultures are discarded.

Yields of RPE cells directly from a single eye range from approximately 250,000 to 1 million cells. The cells are small, round and filled with melanin granules that give the cells a dark black appearance. Upon introduction into culture, cells migrate out from the fragments of RPE sheets that are attached to the flasks. Melanin granules are visible in greater than 95% of the migrating cells and constitute an index of RPE cell purity in the preparation. Morphologically, the RPE cells change from small, round black cells to larger, cuboidal cells, with greatly diminished pigmentation as they spread outward from the RPE tissue fragments. The original morphological appearance is reacquired, upon establishment of culture confluence. At confluence, the 25 cm² culture flask yields approximately 5 million cells. The cells are recovered from the flask by exposure to 0.2% trypsin (radiation sterilized, qualified) for 10 minutes, followed by scraping the cells from the flask surface with a sterile spatula (CoStar, Cat. No. 3008). Scraping is necessary because the cells are very tightly adherent and the extended times necessary for dissociating the cells from the flasks with trypsin digestion alone produces very low cell viability (10% or less). The combination of trypsinization and scraping produce preparations with greater than 90% viability as judged by Trypan Blue dye exclusion.

RPE cells recovered from the flasks are divided into three aliquots and further processed as follows. Aliquot 1 (about 4.5 million cells) and Aliquot 2 (about 0.45 million cells) in 1 ml of antibiotic/antimycotic free-Culture Medium is adjusted to a final concentration of 7.5% with DMSO (Sigma, Cat. No. D2650, qualified for endotoxin and tested in culture) and 20% qualified fetal calf serum. The cells are transferred into cryopreservation vials and frozen in a controlled rate cryopreservation apparatus (Nalge, Cryo-1-C, Cat. No. 5100-001). The vials used are from Corning (Corning, Cat. No. 25704). Aliquot III is utilized for immunoperoxidase staining, immunofluorescent staining and immunohistochemistry staining for known markers for RPE cells. The cells are plated onto sterile, multi well glass slices coated with Pronectin F, the cells allowed to attach overnight in the culture incubator and then further evaluated for the presence of markers to judge the purity of the RPE cells in culture include the presence of cytokeratin, vesicular dopamine transporter protein, and tyrosine hydroxylase.

### 6.1.2. ELISA Assays of Conditioned Medium

RPE cells were isolated and cultured as described above in section 6.1.1, except that the collagenase utilized was from SigmaType 1a (Cat. No. C-9891 and also two culture media were utilized in different experiments as described. Initially, the cells were placed in either DMEM-F12 culture medium (Gibco, Cat. No. 12440-20 and 1765-021) or in RPMI 1640 (Gibco, Cat. No. 21870-084). Both culture media were supplemented with 2 mM glutamine, 10% fetal calf serum, an antibiotic/antimycotic reagent and acidic FGF (10 ng/ml). The cells were plated in culture flasks coated either with mouse laminin (Gibco, Cat. No. L2020) or with Pronectin F (Protein Technologies, Cat. No. 5002-00, Lot. No. RO117-C). The RPE cells were grown to confluence and passaged in either the DMEM/F12 medium containing 10% fetal calf serum or in RPMI 1640 containing 2% fetal calf serum. When using DMEM/F12, the cells were plated onto flasks coated with laminin. If using the RPMI 1640 medium, the cells were subcultured onto ProNectin coated flasks.

When the RPE cells had reached confluence, the culture media was harvested and stored frozen at −80° C. until assayed for the presence of Fas L by ELISA or bioassay with fetal thymocytes. The ELISA assay protocol includes the following steps. Ninety-six well plates (quality Biologicals, Cat. No. 3791) are coated with anti-human Fas L antibody (Santa Cruz Biotech, Cat. No. SC-956 or Pharmingen, Cat. No. 65431a) by adding 100 ul/well of a stock antibody solution (10 ug antibody/ml) and allowing coating to proceed overnight in the cold room. The 96-well plates are then washed three times with 0.5 ml of phosphate buffered saline (PBS, Irvine Scientific, Cat. No. 9240) containing 0.05% Tween (Tween-20, BioRad, Cat. No. 170-6531). Non-specific protein binding was then minimized by coating unbound sites on the plates with 200 ul of 1% bovine serum albumin (Amersham, Cat. No. RPN 412) in PBS. After standing for 2 hrs at 37° C., the blocking solution is decanted and the wells washed once with 0.5 ml of PBS-Tween. The plates prepared as above were further incubated either with Fas L peptide (Santa Cruz Biotech, Cat. No. SC 956 L, 0-100 ng in 100 ul PBS to generate a standard curve) or with 100 ul of conditioned medium harvested from RPE cell (passage 0, through passage 9). After the Fas L peptide or Fas L in the conditioned medium had bound to the plates for 1 hour at room temperature, the plates were washed three times with PBS-Tween. A second, biotinylated, anti-human Fas L antibody was added to form a sandwich (Biotinylated NoK-1 antibody, Pharmingen, Cat. No. 65322, 100 ul of a 5 ug/ml solution). After binding for 1 hr at room temperature, the unbound antibody was washed off the plates with 3 washes of PBS-Tween. Avidin-horse radish peroxidase solution (ABC Vectrastain, Vector Labs, Cat. No. PK-6100) was then added at 50 ul per well and the binding to biotin-antibody performed by incubation for 30 min at room temperature. The unbound avidin-horse radish peroxidase was removed with three washes of PBS-Tween. One-hundred ul of OPD solution was then added for color development. The OPD (orthophenylenediamine, Sigma, Cat. No. P6662) solution was prepared by dissolving OPD at 0.5 mg/ml in 50 mM phosphate-citrate buffer, pH 5.0 (Sigma, Cat. No. P-4922) containing 1% hydrogen peroxide. After suitable color development had occurred by incubation of the plates at room temperature, the reaction was stopped by the addition of 2 N sulfuric acid solution (Sigma, Cat. No. S. 1526). The absorption of the plates was determined on a Bio-Tek Microplate BioKinetics plate reader (Model EL 340) using a 490 nm filter.

Standard curves were generated using the N-terminal 22 amino acid synthetic peptide of Fas L (SC0567). The peptide was added to culture medium with supplements identical to those used for cell culture) to generate a standard curve, with 0-60 ng Fas L peptide per 200 ul of reaction medium.

### 6.1.3. Fas L Induced Apoptosis Bioassays

To determine whether the cross reacting material was capable of inducing apoptosis, as is the case with intact Fas L (surface bound or free), bioassays were performed.

Apoptosis of lymphocyte populations is inducible upon the interaction of cell surface bound Fas with its ligand, Fas L. Induction of apoptosis requires, however, that the lymphocytes be activated (i.e., as by treatment with anti CD3 antibodies for T cell subsets). Fetal thymocytes are in a high state of activation in vivo and can be used for apoptosis studies in vitro, without the requirement for activation.

The experimental protocol with fetal thymocytes was as follows. 7.5 million freshly isolated human fetal thymocytes (ABR, Inc.) were incubated in 5 ml of fresh medium or RPE cell conditioned-medium (DMEM/F12 medium containing 10% fetal calf serum) for 6-12 hours. RPE cell conditioned medium used in the assays had been previously screened for Fas L content by ELISA assays and contained Fas L cross reacting material in a concentration range of 0-13 ng/100 ul of conditioned medium.

Following the incubation, the cells were spun down in a centrifuge (5 min at 100 rpm) and the cell pellet fix, permeabilized and stained and using the APO-DIRECT™ kit provided by Pharmingen. Staining involved the use of propidium iodide for total DNA content and the use of FITC-dUTP and terminal deoxynucleotide transferase to label DNA chain breaks. Two color FACS analyses were performed to quantitate I propidium iodide and FITC-dUMP fluorescence, using a Beckton-Dickinson FACS scan cell sorter. Electronic gating was utilized to eliminate cell aggregates. The data presented therefore relates to single cells.

### 6.2. RESULTS

### 6.2.1. Results of the ELISA Assays

Standard Curves were generated using the N-terminal 22 amino acid synthetic peptide of Fas L (Sc9567). The standard curve data generated are indicated below.

| SC9567 Conc. | Absorbancy | | Standard |
|---|---|---|---|
| ng/200 ul | (490 nM) | Average | Dev. |
| 0 | 0.044, 0.046 | 0.045 | 0.001 |
| 2.5 | 0.072, 0.076 | 0.074 | 0.002 |
| 5.0 | 0.161, 0.121 | 0.143 | 0.029 |
| 10.0 | 0.151, 0.197 | 0.174 | 0.033 |
| 60.0 | 0.517, 0.629 | 0.573 | 0.079 |

Using the values for the standard curve above, the values of Fas L cross reacting material in RPE cell-conditioned medium (RPE CM) (values per 100 ul aliquot) were calculated, using the Santa Cruz anti-Fas L antibody and are listed below.

| | Mean | Standard | |
|---|---|---|---|
| Sample | Absorbancy | Deviation | ng Fas L |
| Analyzed | (490 nm) | (Absorption) | (Per 100 ul) |
| Control medium | 0.063 | 0.001 | 2.5 |
| RPE CM | 0.091 | 0.004 | 5.2 |
| (DMEM/F12, P0 | | | |
| RPE CM | 0.114 | 0.017 | 6.5 |
| (DMEM/F12, P1) | | | |
| RPE CM | 0.115 | 0.02 | 6.6 |
| (DMEM/F12, P3) | | | |
| RPE CM | 0.139 | 0.033 | 8.0 |
| (RPMI 1640, PO) | | | |
| RPE CM | 0.292 | 0.044 | 17.0 |
| (RPMI 1640, P1) | | | |
| RPE CM | 0.228 | 0.031 | 13.0 |
| (RPMI 1640, P2) | | | |
| RPE CM | 0.157 | 0.011 | 9.0 |
| (RPMI 1640, P0) | | | |
| RPE CM | 0.130 | 0.013 | 7.5 |
| RPE CM | 0.202 | 0.004 | 12.0 |
| (RPMI 1640, P1) | | | |
| RPE CM | 0.167 | 0.006 | 9.6 |
| (DMEM/F12, P0) | | | |

ELISA assays of late passage RPE cells grown in RPMI 1640+2% or +10% fetal calf serum or DMEM/F12+10% fetal calf serum are shown below. In the former case, the cells were plated on Pronectin F coated flasks, whereas in the latter case, the cells were plated on mouse laminin. Calculations of the mass of Fas L are normalized at the absorbancy at 490 nm for the SC9567 Fas L peptide value at 5 ng/assay. A control for medium not exposed to RPE cells is also included. The results are as follows:

Cells grown in DMEM/F12 + 10% FCS

| | Mean | Standard | |
|---|---|---|---|
| | Absorbance | Deviation | Fas L |
| Sample | (490 nm) | (Absorbancy) | (Ng/100 ul) |
| SC9567, 5 ng | 0.461 | 0.001 | 5.0 |
| Control Medium | 0.063 | 0.007 | 0.7 |
| RPE CM, P4 | 0.870 | 0.124 | 9.4 |
| RPE CM, P5 | 0.544 | 0.101 | 6.0 |
| RPE CM, P6 | 0.442 | 0.120 | 4.7 |
| RPE CM, P7 | 0.136 | 0.025 | 1.5 |
| RPE CM, P8 | 0.529 | 0.160 | 5.7 |
| RPE CM, P9 | 0.793 | 0.191 | 8.6 |

Cells Grown in RPMI 1640 + 2% FCS

| | Mean | Standard | |
|---|---|---|---|
| | Absorbance | Deviation | Fas L |
| Sample | (490 nm) | (Absorbancy) | (Ng/100 μl) |
| SC9567, 5 ng | 0.461 | 0.001 | 5.0 |
| RPMI Control | 0.085 | 0.012 | 0.9 |
| Medium | | | |
| RPE CM, P4 | 0.628 | 0.087 | 7.0 |
| RPE CM, P5 | 0.395 | 0.039 | 4.3 |
| RPE CM, P6 | 0.427 | 0.066 | 4.6 |
| RPE CM, P7 | 0.379 | 0.086 | 4.1 |
| RPE CM, P8 | 0.524 | 0.026 | 5.7 |

Cells grown in RPMI 1640 + 10% FCS

| | Mean | Standard | |
|---|---|---|---|
| | Absorbance | Deviation | Fas L |
| Sample | (490 nm) | (Absorbancy) | (Ng/100 μl) |
| SC9567, 5 ng | 0.461 | 0.001 | 5.0 |
| RPMI Control | 0.049 | 0.000 | 0.5 |
| Medium | | | |
| RPE CM, P4 | 0.653 | 0.070 | 7.0 |
| RPE CM, P5 | 0.418 | 0.120 | 4.5 |
| RPE CM, P6 | 0.452 | 0.039 | 5.8 |
| RPE CM, P7 | 0.425 | 0.018 | 4.6 |
| RPE CM, P8 | 0.359 | 0.073 | 4.0 |

### 6.2.2. Evaluation of the RPE Conditioned Medium for Apoptosis-inducing Activity Against Thymoctyes

The results described above indicate that the RPE cells release material into the culture medium that is immunologically related to the N-terminal peptide of Fas ligand in assays with the antibody preparation from Santa Cruz BioTech. Similar experiments were performed using anti-Fas L antibody obtained from Pharmingen, which confirmed the presence of Fas L cross-reacting material.

Negative control or positive control cells are treated with FITC-dUTP in the presence of TdT enzyme. This leads to the incorporation of FITC-dUTP into the DNA fragments found in apoptotic cells. Cells are then stained with propidium iodide and analyzed on a Beckton Dickinson FACSCAN™. The presence of apoptotic cells is demonstrated by increased fluorescence intensity as apoptotic cells are clearly labeled with FITC (yellow-green cells), while non-apoptotic cells show only the red staining of propidium iodide.

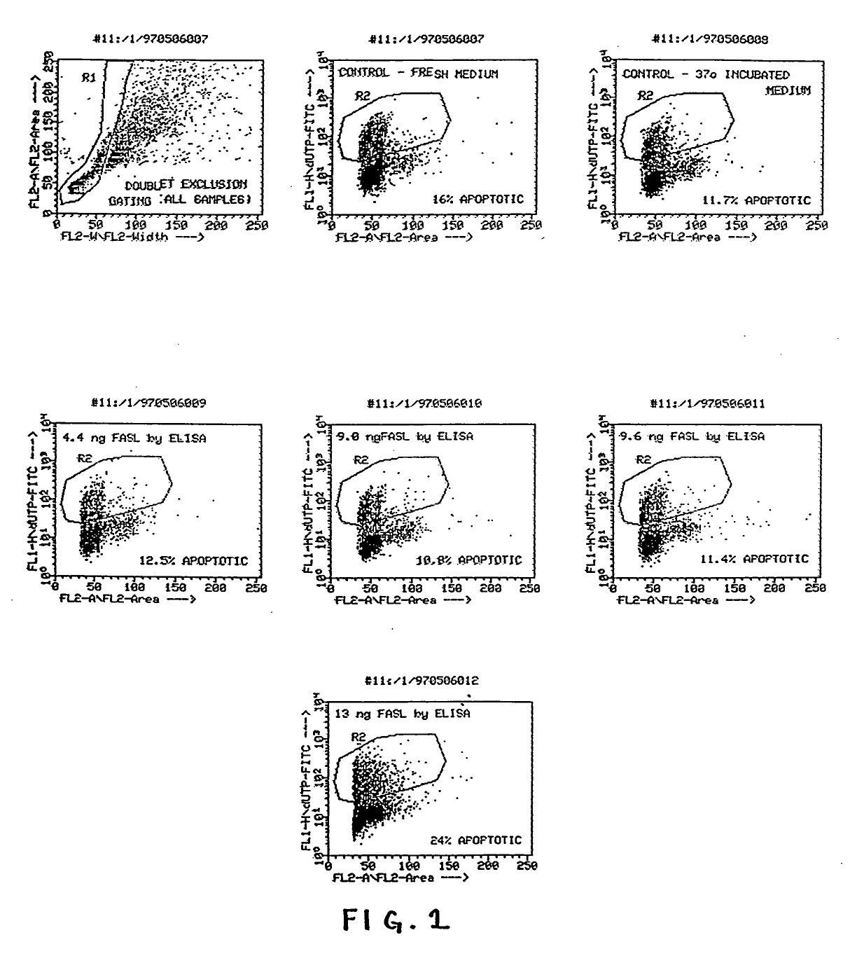

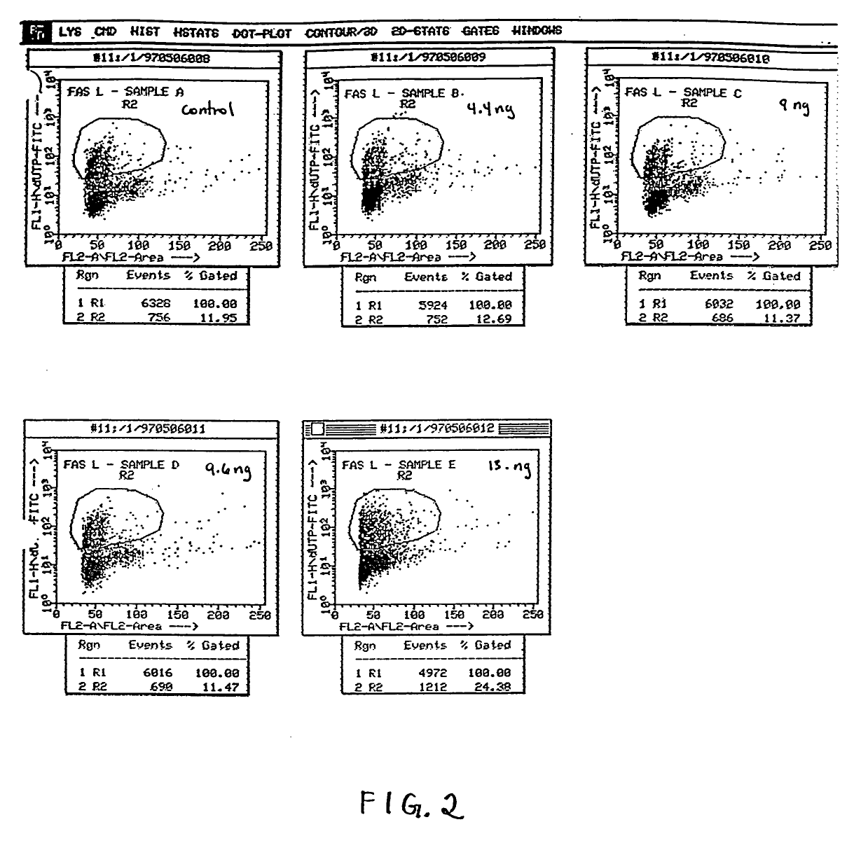

The results of the FACS analysis are presented in FIG. 1 and are summarized in the accompanying table inserts of FIG. 2. To briefly summarize, apoptosis in the thymocytes incubated in fresh medium (not exposed to RPE cells) was approximately 12%. No indication of apoptosis was seen until the Fas L concentration of the RPE CM had reached its highest value, i.e., 13 ng/100 ul of conditioned medium. At this point, the apoptotic value had risen to 24% or to approximately twice that of the control medium. The failure to see apoptosis generated at lower Fas L concentrations may indicate that the Fas L is significantly degraded or may indicate that the apoptosis inducing activity is marginal as the free ligand until high concentrations are attained.

1-24. (canceled)

25. A pharmaceutical composition comprising retinal pigment epithelial (RPE) cells, a non-RPE cell population, and a pharmaceutically acceptable carrier, wherein said non-RPE cell population comprises insulin-producing β cells.

26. The composition of claim 25 wherein said insulin-producing β cells are pancreatic islet of Langerhans cells.

27. A compartmentalized kit adapted to receive a first container adapted to contain retinal pigment epithelial (RPE) cells and a second container adapted to contain a non-RPE cell population, wherein the non-RPE cell population comprises insulin-producing β cells.

28. The compartmentalized kit according to claim 27 wherein the insulin-producing β cells are pancreatic islet of Langerhans cells.

29. The composition of claim 25 wherein said RPE cells are attached to a matrix.

30. The composition of claim 25 wherein cells of said non-RPE cell population are attached to a matrix.

31. An article of manufacture, comprising:
a packaging material;
retinal pigment epithelial (RPE) cells containing within said packaging material;
a non-RPE cell population contained within said packaging material, wherein the non-RPE cell population comprises insulin-producing β cells; and
wherein said packaging material contains a label that indicates that said RPE cells can be used for facilitating survival of an allogeneic graft of the non-RPE cell population in a mammal.

32. The article of manufacture according to claim 31, wherein the insulin-producing β cells are pancreatic islet of Langerhans cells.
